(19) United States
(12) Patent Application Publication
(10) Pub. No.: US 2019/0291910 A1
(43) Pub. Date: Sep. 26, 2019

(54) Method and Filling Machine for Filling Packages Open on One Side with a Processing Movement Different from the Transport Direction

(71) Applicant: SIG Technology AG, Neuhausen am Rheinfall (CH)

(72) Inventors: Hanno Geissler, Krefeld (DE); Richard Heuser, Herzogenrath (DE); Markus Klaus, Eschweiler (DE); Franz-Josef Laven, Heinsberg (DE); Klaus Naber, Kempen (DE); Frank Ott, Neuss (DE); Thomas Patzer, Juelich (DE); Christian Weiler, Geldern (DE)

(21) Appl. No.: 16/464,832

(22) PCT Filed: Nov. 30, 2017
(86) PCT No.: PCT/EP2017/081084

§ 371 Date: May 29, 2019

(30) Foreign Application Priority Data
Nov. 30, 2016 (DE) 10 2016 123 144.4

(51) Int. Cl. B65B 55/10; B65B 3/04; B65B 3/02; B65B 3/22; B65B 43/50; B65B 43/60; B65B 55/02; B65B 55/06

(52) U.S. Cl. CPC B65B 55/10; B65B 3/04; B65B 3/025; B65B 3/22; A61L 2/07; B65B 43/60; B65B 55/027; B65B 55/06; B65B 43/50

(57) ABSTRACT

A method is described for filling packages open on one side with flowable products in a filling machine. In order for the packages open on one side to be processed reliably, it is provided that hot air, drying air, sterilising agent and/or flowable product is introduced via at least one nozzle into packages open on one side, that the packages are moved from a starting position, before the introduction of the air, agent, and/or flowable product, into an end position, after the introduction of the air, agent, and/or flowable product, and that the packages, during the introduction of the air, agent, and/or flowable product, are moved along a straight or continuously curved transport direction defined by the starting position and the end position and are moved in a processing movement overlaying the transport direction and extending at least in sections transversely to the transport direction.

The invention relates to a method for filling packages open on one side, in particular cardboard composite packages, with flowable products, in particular food, in a filling machine. Furthermore, the invention relates to a filling machine for filling packages open on one side, in particular cardboard composite packages, with flowable products, in particular foods, preferably according to the method specified before, wherein at least one nozzle is provided for introducing hot air, drying air, sterilising agent and/or flowable product into the packages open on one side and wherein a transport device is provided for transporting the packages open on one side from a starting position, before the introduction of the hot air, the drying air, the sterilising agent and/or the flowable product, into an end position, after the introduction of the hot air, the drying air, the sterilising agent and/or the flowable product, along a straight or continuously curved transport direction defined by the starting position and the end position.

Different filling machines for filling packages open on one side with products, especially in the form of food, are already known. The packages are preferably filled with flowable food. The packages used in particular are packages which are open at an upper side in order to provide an opening for filling the packages. The packages can be, for example, cardboard composite packages which are formed from a packaging material in the form of a laminate comprising a cardboard layer and outer, in particular thermoplastic, plastic layers, for example made of polyethylene (PE). The carton provides the packages with sufficient stability to allow the packages to be easily handled and stacked, for example. The plastic layers protect the cardboard from moisture and allow sealing of the packaging material to form a sealed package. In addition, further layers, such as an aluminium layer, may be provided to prevent diffusion of oxygen and other gases through the package.

The filling of the packages with food is typically performed under sterile conditions. Not only the food to be filled, but also the package has to be sterilised. Thus, the food is usually heated for a certain period of time. First, the packages are generally purged with sterile hot air. A sterilising agent is then introduced into the packages heated thus, which typically is or contains hydrogen peroxide. As the package is preheated, high sterilisation reaction rates are achieved, avoiding the formation of excess condensate in the package. Moisture and residual hydrogen peroxide are then removed from the package by drying the package with preferably hot and sterile drying air. The filling of the sterile package then takes place.

The device for introducing hot air, drying air, sterilising agent and/or flowable product comprises nozzles for targeted impingement of the packages open on one side with the appropriate medium. The packages are transported by means of a transport device through the filling machine and thereby successively to the corresponding nozzles. In order to prevent damage to the packages and still to transport the packages precisely, the transport devices regularly have carriers or cells for holding the packages during transport. Thus, the packages can be moved through the filling machine indirectly via the carriers or cells. In this case, carriers are to be understood as means on which the packages predominantly stand and are held laterally, while cells are to be understood as means which primarily accommodate the packages to a large extent and/or hold them laterally. In particular, in filling machines in which the processing of the packages takes place substantially in a straight line, the term ‘inline filling machines’ is also used to distinguish these filling machines from so-called rotary units, cell chains are typically used to transport the packages, in which a plurality of cells for receiving the packages are provided along a revolving chain. The cells may also be designed in the form of holders which hold the packages by lateral contact. This is particularly the case with rotary units in which the packages are transported from the starting position and the end position along a rotating carousel along a constant bent curve. The radius of the curve typically corresponds to the distance between the packages and the axis of rotation of the carousel of the rotary unit.

During impingement of the packages with hot air, drying air, sterilising agent and/or flowable product, the packages can be transported past under the corresponding nozzles. This is carried out starting from a starting position of the packages before they are impinged with hot air, drying air, sterilising agent and/or flowable product up to an end position after they are impinged with hot air, drying air, sterilising agent and/or flowable product, and takes place regularly in a straight line or in an arc with constant curvature, that is in a circular arc. The transport of the packages can be effected continuously, if necessary, at a constant speed. If desired, the nozzles may be moved with the packages, such that there is no relative velocity between the packages and the nozzles. However, it is simpler and more cost-effective if the nozzles are provided in a stationary position.

Frequently, the packages are also moved through the filling machine in a timed manner, i.e. step-wise, so that the packages successively occupy certain positions for a certain period of time, in which the packages are stationary impinged via at least one nozzle with hot air, drying air, sterilising agent and/or flowable product. Corresponding filling machines are typically designed as inline units. While processing the packages open on one side with hot air, drying air, and/or sterilising agent or when filling the packages open on one side with flowable product in a rotary unit, the packages are typically moved continuously, but remain, as with the timed operation of the inline unit, for a certain period of time, stationary relative to the at least one nozzle dispensing hot air, drying air, sterilising agent and/or flowable product, which moves at the same speed along at least substantially the same circular arc as the associated package.

In any case, it must be ensured that the packages are sufficiently impinged with hot air, drying air, sterilising agent and/or flowable product to be sufficiently preheated, dried, sterilised and/or filled. On the one hand sufficient hot air, drying air, sterilising agent and/or flowable product must be directed from the nozzle onto/into the packages and on the other hand the package must remain under the nozzle for a sufficiently long time. At the same time, however, filling machines are intended to fill as many packages as possible in the shortest possible time and in the smallest possible space. In addition, the purging of the packages with hot air, drying air and/or sterilising agent or the filling of the packages with flowable product is only conditionally predictable. The currents forming in the packages are to a degree random or chaotic. This leads in part to the formation of undesirable shaded regions that do not come into contact with the hot air, the drying air and/or the sterilising agent to the desired extent, for example because these surfaces are partially covered by inert boundary layers. However, under certain circumstances, this also leads to foaming during filling of the flowable product.

It is therefore the object of the present invention to design and further develop the method and the filling machine of the type mentioned and explained above, in such a way that the packages open on one side can be reliably impinged with hot air, drying air, sterilising agents and/or flowable product reliably with reasonable equipment and procedural complexity at high throughput.

The object is achieved according to claim 1 by a method for filling packages open on one side, in particular cardboard composite packages, with flowable products, in particular food, in a filling machine,
in which hot air, drying air, sterilising agent and/or flowable product is introduced via at least one, in particular stationary, nozzle into packages open on one side,
in which the packages are moved from a starting position, before the introduction of the hot air, the drying air, the sterilising agent and/or the flowable product, into an end position, after the introduction of the hot air, the drying air, the sterilising agent and/or the flowable product, and
in which the packages, during the introduction of the hot air, the drying air, the sterilising agent and/or the flowable product, are moved along a straight or continuously curved transport direction defined by the starting position and the end position and are moved in a processing movement overlaying the transport direction and extending at least in sections transversely to the transport direction.

In addition, the stated object is achieved with a filling machine according to the preamble of claim 9 in that means are provided for moving the packages during the introduction of the hot air, the drying air, the sterilising agent and/or the flowable product in a processing movement overlaying the transport direction and extending at least in sections transversely to the transport direction.

The invention has recognised that it can have a favourable effect on the processing of packages open on one side, if these are not moved exclusively in a straight manner or along an arc of constant curvature, thus a circular arc, from the starting position to the end position and in between are impinged by impinging with hot air, drying air, sterilising agent and/or flowable product. Instead, it is proposed, overlaying with this basic movement in the transport direction of the packages, to provide a processing movement which is performed by each package between the starting position and the end position. This processing movement serves less to actually transport the packages to the end position, but rather to achieve a more reliable processing of the packages with hot air, drying air and/or sterilising agent or to more reliably fill the packages with flowable product. This can in fact be improved if the packages are not merely positioned stationary underneath the nozzle or transported in a simple movement under the nozzle, but are additionally moved during processing or filling, by means of the processing movement.

With regard to the processing movement, no distinction is made in the present case between processing with hot air, drying air and/or sterilising agent and filling with a flowable product, in particular food. Although the package open on one side is not processed in the latter case, the filling of the package open on one side also represents a processing step of a filling machine in a broader sense. For the sake of clarity and to avoid unnecessary repetitions, therefore, no fundamental distinction should be made between the processing movement and the filling movement, depending on whether the packages are processed or filled.

The additional processing movement in addition to the transport movement then ensures that the hot air, the drying air and/or the sterilising agent reaches all intended areas of the package and sufficiently interacts with all these surfaces to achieve a uniform and reliable heating, drying and/or sterilisation. If necessary, an additional processing movement ensures that the foaming is reduced when filling with flowable product. This is achieved, for example, by the fact that the flow of the hot air, the drying air, the sterilising agent and/or the flowable product is redirected in the package in a targeted manner. It is accepted, for example, that during the impingement of the packages with hot air, drying air and/or sterilising agent or during the filling of the packages, the introduction of the corresponding medium is not optimal at least for a major part of the impingement duration or filling time, but if necessary even highly suboptimal. This is then compensated by the processing movement. Thus, for example, different surfaces of the package can be impinged particularly well and other surfaces of the package less well at different times. With completed impingement, however, all surfaces have been impinged very reliably in a suitable manner.

It might be expected that much longer processing of the packages would be required if processing was suboptimal for substantially all of the processing time, and that it would be better in terms of shortening the processing time and increasing the throughput of the filling machine, if during the entire processing step at least virtually optimal processing of the packages were to be effected. However, it has surprisingly been found that due to the somewhat random and chaotic flow behaviour of the hot air, the drying air and/or the sterilising agent in the packages this is not the case. In addition, it has been found that changing filling direction during filling suppresses the foaming particularly effectively, rather than using a particular filling direction based on the package, as is commonly assumed.

The processing movement can be carried out in different ways, as will be explained below. If the processing movement is understood as an overlay to the basic transport direction or transport movement, which is the uniform transport of the packages from the starting position to the end position along a straight line or a uniformly curved arc, the processing movement can be divided, for example, into a movement back and into a movement forth. Thus, the actual package path, which the packages follow, will deviate initially from the basic straight or constantly curved transport direction or transport movement, in order to subsequently revert to the virtual straight or constantly curved packaging path, so that the packages reach the end position after termination of the processing, which is the end position that the packages would reach even with exclusive movement of the packages in the straight or constantly curved transport direction.

In the present case, the term nozzle is preferably understood very generally. A nozzle with significant cross-sectional constriction for producing a significant pressure loss or a greatly widening free jet is not necessarily required. The nozzle is only intended to allow a reliable and targeted feeding of the corresponding medium into the package at a predetermined position. In a particularly simple case, the nozzle may be formed as a straight piece of pipe, which can be aligned very precisely towards the packages.

Furthermore, the terms ‘hot air’ and ‘drying air’ are preferably to be understood very broadly. The appropriate medium is used for preheating and drying, which could also be effected entirely without air, but with another gas or gas mixture. Thus, when the terms ‘hot air’ and/or ‘drying air’ are used, the term ‘air’ is preferably understood generally as a gas or mixture of gases. However, since air is readily available at low cost, it is particularly preferable if actual air is used. Even in practice, air is generally used. Therefore, in the present case for the sake of clarity and to avoid unnecessary repetition, the terms ‘hot air’ and ‘drying air’ are used, even if these media need not be limited to air and could refer in principle to a heating medium or drying medium.

The packages are preferably laminate packages formed from packaging material laminates. In a particularly preferred case, these are cardboard composite packages made of a packing laminate comprising at least one cardboard layer, preferably at least one barrier layer, for example consisting of aluminium, polyamide and/or an ethylene-vinyl alcohol, and outer layers made of a thermoplastic material, in particular polyethylene (PE) or polypropylene (PP). The longitudinal edges of a packaging material blank formed from the packaging material laminate are sealed together to form what is known as a packaging sleeve or packaging wrap. One of the open ends of the packaging sleeve can be folded together and sealed to form a package base. After filling, the still open end of the packaging sleeve can also be closed by folding and sealing into a package top.

For ease of understanding and to avoid unnecessary repetition, the method and the filling machine will be described together below, without distinguishing in detail between the method and the filling machine. However, from the context it will be apparent to those skilled in the art which particular feature is preferred with respect to the method and the filling machine.

In a first particularly preferred embodiment of the method, the packages are moved respectively on a carrier and/or respectively in a cell of a transport device from the starting position to the end position. To facilitate handling of the packages, it may further be preferable for the packages to be transported through the filling machine situated as they are, respectively on a carrier and/or in a cell. Essentially, the carriers or cells allow a reproducible and precise transport of the packages without exposing the packages to excessive stresses that could damage the packages. In order to effect the processing movement of the packages in a simple manner and to improve the processing of the packages in this way, the carriers and/or the cells can be moved in sections in at least one direction transversely to the transport direction, in particular back and forth. The packages, carriers and/or cells are thus initially led away from a virtual packing path, which is defined by the straight or constantly curved transport direction or transport movement, in order to be guided back afterwards to the virtual packaging path as defined by the straight or constant curved transport direction or transport movement. The transport movement and the processing movement can consequently be regarded as movements overlaying between the starting position and the end position.

In order to provide a suitable processing movement between the starting position and the end position, it is advisable to tilt the packages, preferably together with at least parts of the carriers and/or cells. The tilting movement may include a back and forth movement component. For example, the packages may be arranged in both the starting position and the end position in the same upright orientation. In order to be able to provide the processing movement in a simple and reliable manner, it may further be appropriate to tilt the packages, preferably together with at least parts of the carriers and/or cells, in a plane parallel to the transport direction and/or in a plane perpendicular to the transport direction.

For a suitable processing movement between the starting position and the end position, it may alternatively or additionally be provided that the packages, preferably together with at least parts of the carriers and/or cells, are moved transversely to the transport direction. This movement can also be subdivided into partial movements back and forth. Thus, the packages can be moved away from the virtual packaging path and back again along the straight or constantly curved transport direction. In this context, it is procedurally and structurally simple if the packages, preferably together with at least parts of the carriers and/or cells, are processed in a horizontal plane and/or in a vertical plane. This also increases the overall reliability of the processing.

In order to be able to provide the processing movement in a structurally simple and procedurally reliable manner, alternatively or additionally, a part of the carriers and/or the cells can be moved from the starting position into the end position at least substantially exclusively in the straight or constantly curved transport direction, while another part of the carriers and/or the cells performs the processing movement. The packages are then associated with the other part performing the processing movement. This can be done for the sake of simplicity, so that the packages stand on the appropriate part or are held by the appropriate part. Thus, on the one hand less mass must be moved in the processing movement, and it is possible to achieve in a very simple manner an overlay of the transport movement along the straight or constantly curved transport direction on the one hand and the processing movement deviating therefrom on the other.

In order to achieve the simplest possible and reliable adjustment of at least a part of the carriers and/or the cells for effecting the processing movement, at least one curved rail and/or at least one cam can be provided. The curved rail and/or the cam can then come into positive contact with the package between the starting position and the end position. This positive contact preferably influences the movement direction of the packages and imparts the processing movement to the packages.

As an alternative or in addition to the curved rail and/or the cam, at least one part of the carriers and/or cells can also be exposed to a magnetic field between the starting position and the end position so as to impart a processing movement to the packages at least in sections in a simple and, if necessary, contactless manner.

Regardless of how the adjustment of parts of the carriers and/or the cells is achieved for imparting at least parts of the processing movement to the packages, this needs not to apply equally to both movement sections back and forth. For reasons of simplicity, deflection of at least parts of the carriers and/or of the cells can be effected in the manner described, while the adjustment takes place back to the starting position autonomously, namely perhaps by the parts of the carriers and/or cells being interconnected via a spring means. The deflection of the parts of the carriers and/or the cells can then take place against the restoring force of the at least one spring means, while the subsequent return of the deflection is effected by the restoring force of the at least one spring means.

In a first particularly preferred embodiment of the filling machine, the transport device has carriers and/or cells for holding and moving the packages from the starting position to the end position. The carriers and/or cells can preferably be transported as such on the carriers and/or with the cells through the filling machine. In addition, the carriers and/or cells for effecting the processing movement of the packages can be adjusted at least in sections in at least one direction transverse to the transport direction, be it provided as straight or constantly curved. The processing movement can be understood as a movement back and forth with respect to the virtual packaging path resulting from the straight or constantly curved transport direction.

For imparting the processing movement to the packages, at least one means for tilting the packages, preferably together with at least parts of the carriers and/or cells, may be provided. The means for tilting the packages, preferably together with at least parts of the carriers and/or cells, can be designed for the sake of simplicity so that the packages can be tilted in a plane parallel to the transport direction and/or in a plane perpendicular to the transport direction, if necessary back and forth.

Alternatively or additionally, however, it is also possible to provide at least one means for moving the packages, preferably together with at least parts of the carriers and/or cells, transversely to the transport direction. This then provides a simplification of the process or apparatus, if the means for moving the packages, preferably together with at least parts of the carriers and/or cells, are formed in a horizontal plane and/or in a vertical plane.

For the overlaying of a transport movement along the straight or constantly curved transport direction with the processing movement provided transverse thereto it is expedient for the sake of simplicity when the carrier and/or cells are formed in at least two parts. One part of the carriers and/or cells can then follow the straight or constantly curved transport direction, while the processing movement is imparted to another part of the carriers and/or cells. In order to achieve this in a structurally simple and reliable manner, the two parts can be mutually displaceable and/or tiltable connected, in particular with at least one rail system, at least one joint and/or at least one spring means. So that a simple and reliable transport of the packages can be ensured, the parts of the carriers and/or cells are connected to one another, for example, in the manner described.

If at least one curved rail and/or at least one cam is provided, the curved rail and/or the cam may be provided for positive contact with at least one part of the carriers and/or cells between the starting position and the end position. By means of the corresponding design of the curved rail and/or of the cam, the positive contact can then ensure that the desired processing movement is imparted to the corresponding part of the cells and/or the carriers and thus to the packages, specifically overlaid on the transport movement, which is defined by the straight or constantly curved transport direction between the starting position and the end position.

A contactless imparting of the processing movement on the packages can preferably be effected with a device, in particular an electrical device, which generates a magnetic field by which the carriers and/or the cells are moved at least in parts. In accordance with the orientation of the magnetic field with respect to the transport direction, it is thus possible to effect targeted deflections, in particular, of the parts of the carriers and/or cells which conduct the packages with respect to the transport movement.

The invention will subsequently be explained in more detail with reference to a drawing depicting only exemplary embodiments. In the drawing:

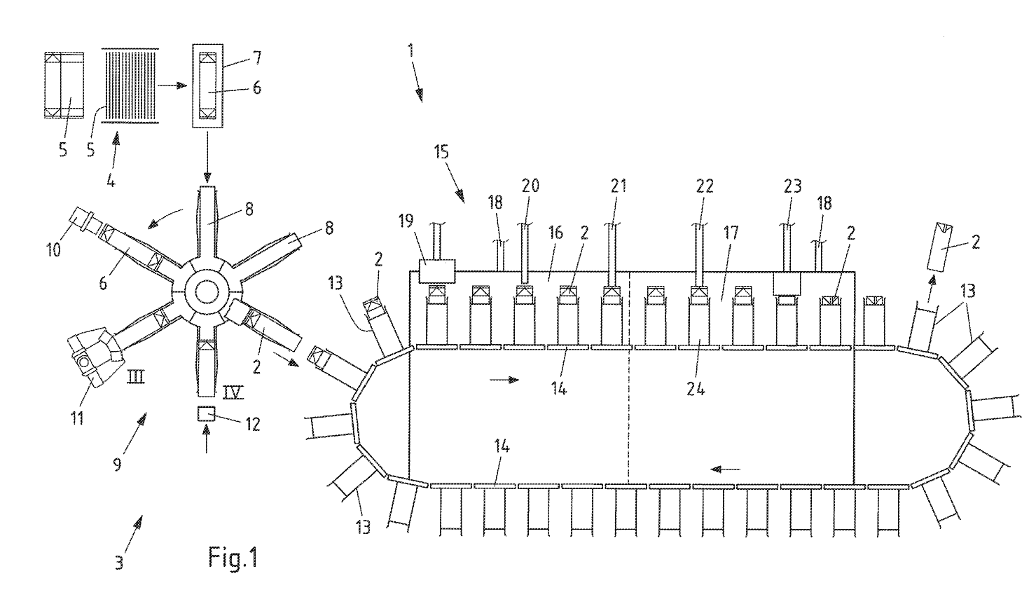

FIG. 1 shows a filling machine according to the invention for carrying out the method according to the invention in a schematic side view,

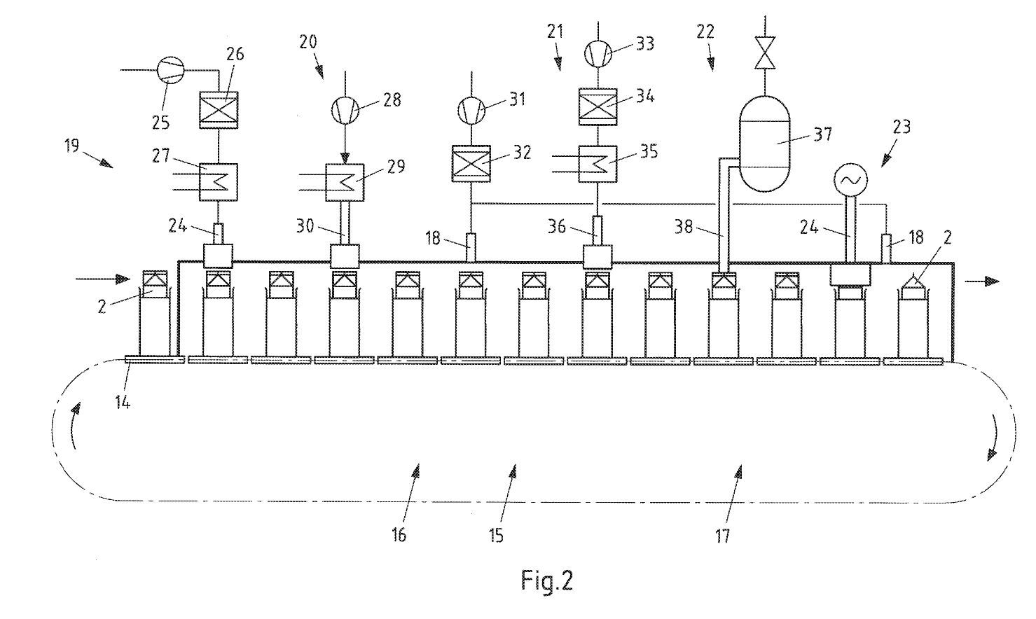

FIG. 2 shows a detail of the filling machine from FIG. 1 in a schematic side view,

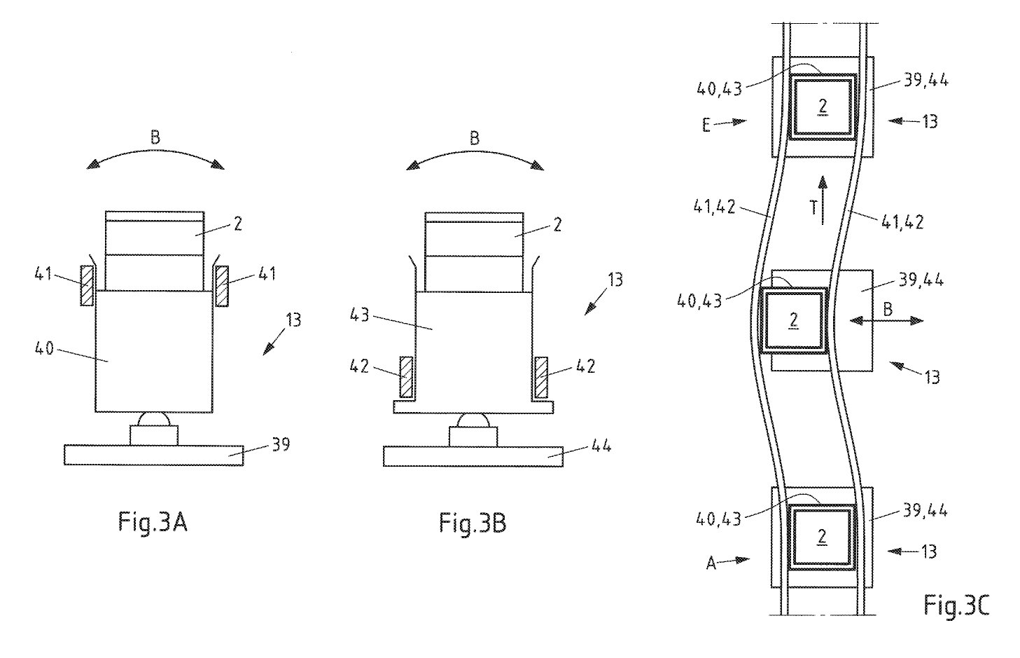

FIG. 3A-C shows overlays of a processing movement and a transport direction between a starting position and an end position in an inline filling machine in schematic views from the side and from above

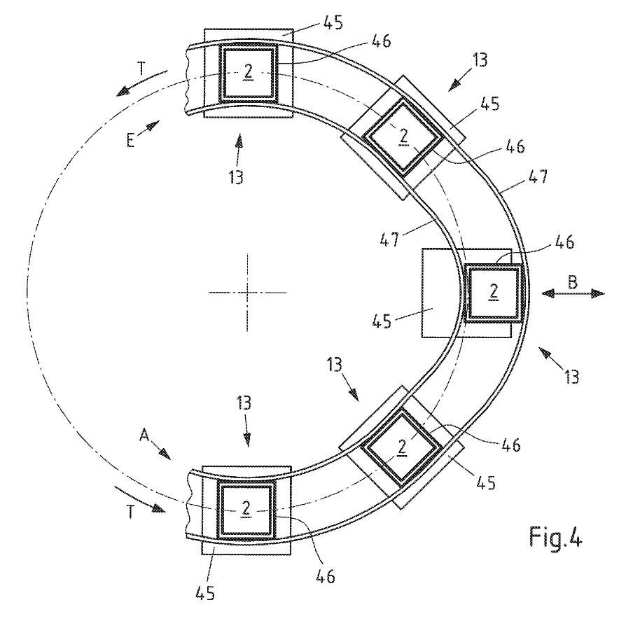

FIG. 4 shows overlays of a processing movement and a transport direction between a starting position and an end position in a rotary filling machine in a schematic view from above,

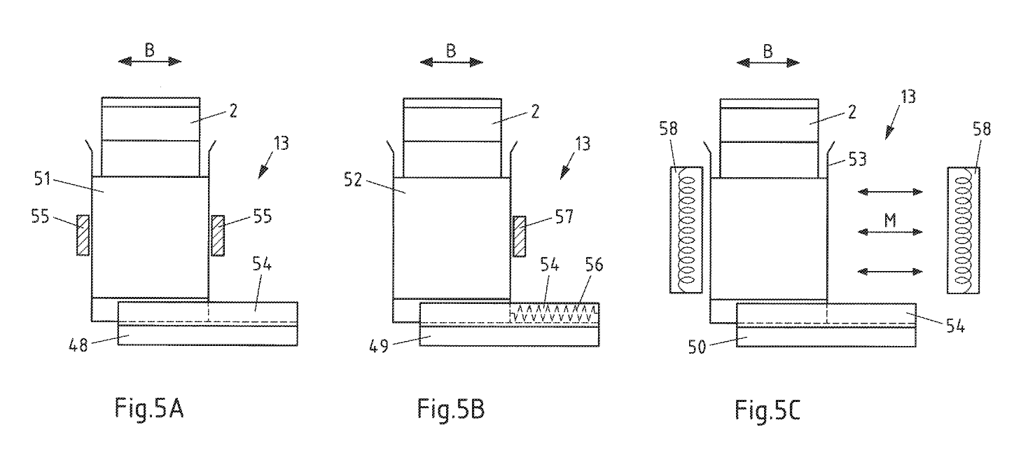

FIG. 5A-D shows alternative overlays of a processing movement and a transport direction between a starting position and an end position in an inline filling machine in schematic views from the side and from above,

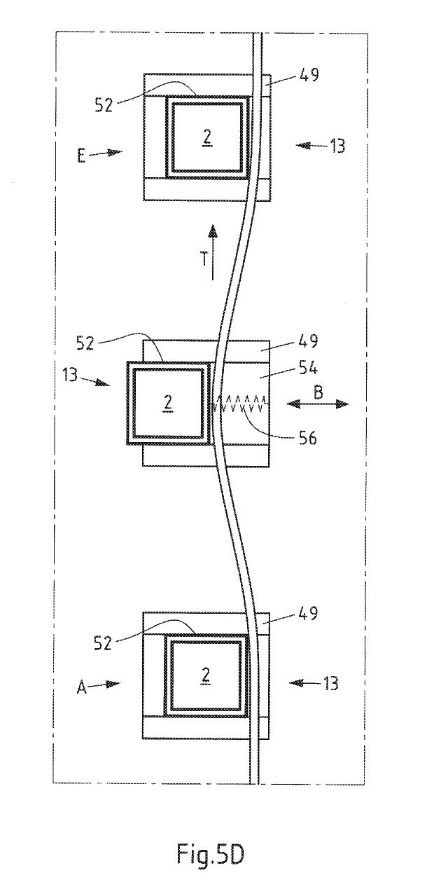

FIG. 6A-B shows an overlay of a processing movement and a transport direction during timed operation of an inline filling machine at two different points in time in a schematic plan view, and

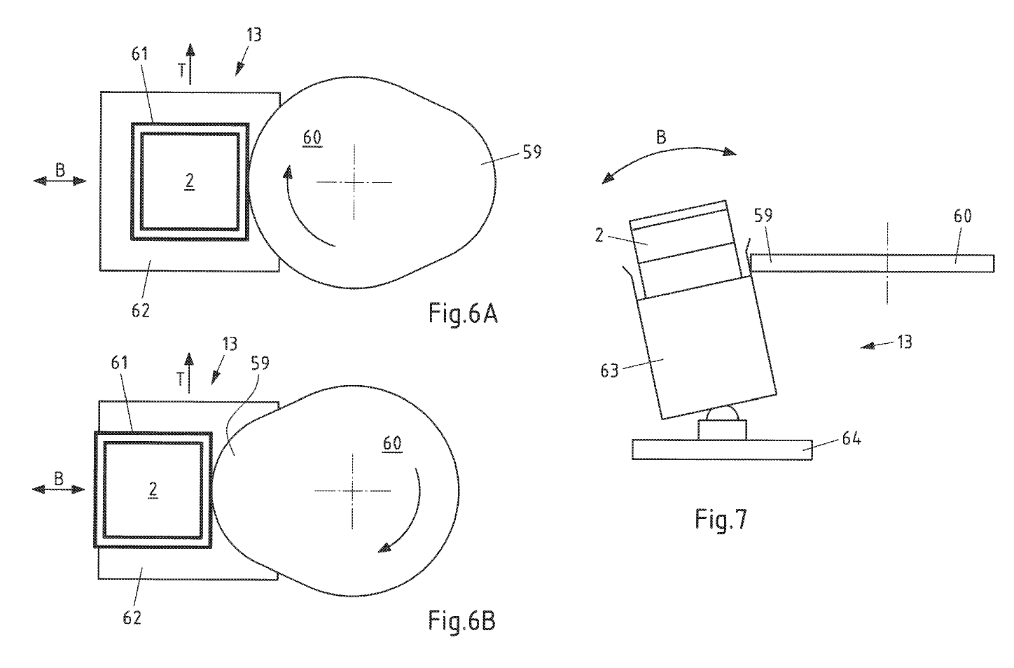

FIG. 7 shows an alternative overlay of a processing movement and a transport direction during timed operation of an inline filling machine in a schematic side view.

FIG. 1 shows a filling machine 1 for filling packages 2, in particular with flowable foods, which in this case comprises a device 3 for shaping the packages 2 to be filled. In principle, however, it is also possible for the filling machine to be already supplied with packages 2 open on one side, which are to be filled. The illustrated and in this respect preferred filling machine 1 also has a number of parallel processing lines, of which only one processing line is shown in FIG. 1. Each processing line is assigned a bundle 4 of package blanks 5, whose longitudinal edges are sealed together and so form packaging sleeves 6 open on both sides. The packaging sleeves 6 are unfolded and pushed onto a mandrel 8 of a mandrel wheel 9 by a feed device 7.

The mandrel 9 is cyclically, that is incrementally, rotated anti-clockwise. The packaging sleeves 6 are processed in different positions. First, an edge is heated by a heating unit 10 using hot air and then pre-folded in a prefold 11 to then seal the prefolded edge to form a base using a press 12. Thus, a sealed package 2 open on one end and tightly sealed on the other is obtained, which is transferred to a cell 13 of a transport device 14. In the illustrated and in this respect preferred filling machine 1, the cells are transported continuously, i.e. without stopping in specific positions, through the filling machines. If necessary, the cells are thereby accelerated and/or decelerated or the transport of the cells by the filling machine 1 is carried out at a constant speed.

The package 2 is transported by means of the transport device 14 through an aseptic chamber 15, which is successively subdivided into a sterilisation zone 16 and a filling and sealing zone 17. The transport of the packages 2 does not have to be straight but can also take place in at least one arc or even in a circle, depending on whether the filling machine 1 is an inline or rotary unit. The aseptic chamber 15 is rinsed with sterile air via corresponding sterile air connections 18 under a slight positive pressure in order to prevent the entry of germs.

Sterile hot air is first blown into the open-topped packages 2 by a preheating device 19 in order to preheat the packages 2. Subsequently, in a sterilising device 20, a sterilising agent is injected into the packages 2, which reacts more strongly in the preheated packages 2 and condenses less. Hydrogen peroxide is particularly suitable as a sterilising agent. The hydrogen peroxide may be introduced together with steam and/or air into the package 2 open on one side. After sterilising the packages 2, the interior of the packages 2 is dried in a drying device 21 by impinging with drying air. The drying air is preferably hot and sterile.

After passing from the sterilisation zone 16 into the filling and sealing zone 17, the packages 2 open on one side are positioned below a filling device 22 and filled with a flowable product in the form of a food. The filled packages 2 are then closed with a closing device 23 by folding the upper open area of the package 2 and sealing it. The sealed packages 2 are then removed from the cells 13 of the transport device 14 and if needed further processed. The now empty cells 13 of the transport device 14 are moved further towards the mandrel wheel 9 in order to receive more packages 2 there.

In FIG. 1, the preheating device 19, sterilising device 20, drying device 21 and filling device 22 are shown only schematically for the sake of better clearness and clarity. Therefore in FIG. 2, the aseptic chamber 15 is shown again in detail. The illustration of FIG. 2 is also very schematic, but in particular the nozzles of the preheating device 19, sterilising device 20, drying device 21 and filling device 22, with which the hot air, the drying air, the sterilising agent or the flowable product are directed towards the packages, are shown larger.

In the aseptic chamber 15 in FIG. 2, a preheating device 19, which has a nozzle 24 for introducing hot air into the packages 2, is initially provided in the sterilisation zone 16. In the illustrated and in this respect preferred filling machine 1, the hot air is suctioned via a compressor 25 from the environment and filtered by means of a filter 26, in order to separate microorganisms and so to sterilise the air. Subsequently, the sterile air is heated in a heating device 27. The packages 2 open on one side are transported past continuously under the stationary nozzle 24 of the preheating device 19.

Thereafter, the packages 2 arrive in a sterilising device 20, in which a sterilising agent, which is an aqueous hydrogen peroxide solution in the illustrated and in this respect preferred filling machine 1, is supplied via a pump 28 and vaporised in a vaporiser 29. The mixture of hydrogen peroxide and steam, which still can be supplied with air, is introduced via a nozzle 30 into the packages 2 transported past underneath.

In the region downstream of the sterilising device 20, sterile air is directed via sterile air connections 18 into the sterilisation zone 16, which can alternatively or additionally also take place at other locations of the aseptic chamber 15. For this purpose, air is suctioned in via a compressor 31 from the environment and pressed through a filter 32, in which all microorganisms are separated to sterilise the air. In the subsequent drying device 21, the sterile packages are dried with hot drying air, which is also suctioned in via a compressor 33 and sterilised in a filter 34. The thus sterilised air is heated in a heating device 35 and directed into the packages 2.

The dried packages 2 are then transferred to the filling and sealing zone 17, where the packages 2 are filled with the product in the form of a food in the next two positions of the transport device 14. The product is supplied in the filling device 22 via a reservoir 37 and is directed into the packages 2 via a nozzle 38. The filled packages 2 are then closed by a closing device 23 by sealing the upper edge and transported out of the aseptic chamber 15.

In the illustrated and in this respect preferred filling machine 1, so called empty positions are provided between individual processing positions along which packages 2 are transported without being processed, while other packages 2 are processed in one of the previously described ways. The arrangement and the number of empty spaces can be chosen in different ways, without this being of particular importance here. Incidentally, alternative preheating devices 19, sterilisation devices 20, drying devices 21 and/or filling devices 22 may additionally have additional nozzles in order to process the packages 2 with the corresponding media and/or to fill them with product. Then, the processing and/or filling of the packages 2 is carried out successively with at least two nozzles in order to extend the processing time or to make the processing more reliable.

When processing the packages 2 with hot air, drying air and sterilising agent as well as when filling the packages, the packages are moved in the preheating device 19, the sterilising device 20, the drying device 21 and the filling device 22 from a starting position before processing to an end position after processing. This movement can be understood in any case as a transport movement, which runs in a straight transport direction in a filling machine 1 designed as an inline filling machine but could also run in a circular arc in a filling machine designed as rotary unit. In addition, in the preheating device 19, the sterilising device 20, the drying device 21 and/or the filling device 22, a processing movement overlaying the transport movement along the transport direction can also be imparted on the packages 2. This ultimately leads to the package following a packaging path, which depends both on the transport direction and on the processing movement.

FIGS. 3A-C show schematic examples of how a processing movement B in the form of a tilting movement can be overlaid on a straight transport movement along the transport direction T of the packages 2. The packages 2 are moved on the one hand in a linear transport movement from the starting position A to the end position E, in particular in a filling machine 1 designed as an inline unit, while the packages 2 are tilted transversely to the transport direction T back and forth. In FIG. 3A, a two-part cell 13 for receiving the packages 2 is shown, which comprises a lower part 39 which is moved in the transport direction T. On the lower part 39, the upper part 40 is provided pivotably relative to the lower part 39. The upper part 40 also receives the package 2 and is guided along one of two curved rails 41, which engage relatively high up against the upper part 40 of the cell 13 and during transport of the package 2 from the starting position A into the end position E come into positive contact with the cells 13. Thus, the package 2 is tilted back and forth transversely to the transport direction T by way of the processing movement B, while hot air, drying air, sterilising agent and/or flowable product are introduced into the package 2 open at the top. Alternatively, as shown in FIG. 3B, curved rails 42 may also be provided, which do not come into positive contact with the upper part 43 of the cells 13 laterally, but rather in the vertical direction. In the embodiment shown in FIG. 3B, the curved rails 42 press down alternately on opposite sides of the upper part 43 of the cells 13 so as to cause the package 2 to tilt in a direction transverse to the transport direction T and back. The movement of the packages 2 for the cases shown in FIGS. 3A-B is shown in a schematic plan view from above in FIG. 3C. While the lower part 39, 44 of the cell 13 is moved from the starting position A to the end position E in a straight line in the transport direction T, the upper part 40, 43 of the cell 13, together with the package 2 incorporated therein, is tilted to the side and back by the curved rails 41, 42 provided laterally of the cells 13.

FIG. 4 shows a transport of the cells 13 and the packages 2 for a filling machine in the form of a rotary unit. The lower part 45 of the cell 13 is thereby moved from the starting position A to the end position E along a circular arc in the constantly curved transport direction T. In between, the upper part 46 of the cell 13 and the package 2 incorporated therein, in addition to the transport movement in the transport direction T, are imparted with a processing movement B in the form of a tilting to the side and back again. This processing movement B is effected by the upper part 46 of the cell 13 being guided along curved rails 47 provided laterally, as has already been shown in principle, for example in FIGS. 3A-B.

FIGS. 5A-D show schematically how, during the transport from the starting position A to the end position E, the transport movement along a straight transport direction T of the packages 2 is overlaid with a processing movement B. In this case, the cells 13 receiving the packages 2 are formed in two parts. The lower part 48-50 of the cells 13 is transported in the transport direction T in the transport movement, while the upper part 51-53 of the cells 13, together with the packages 2 incorporated therein, are moved back and forth transversely to the transport direction T in the processing movement B. For this purpose, the parts 48-53 of the illustrated cells 13 are connected to each other via a rail system 54, which allows the displacement of the parts 48-53 transversely to each other but locks the parts 48-53 of the cells 13 to each other in the transport direction T. In the case shown in FIG. 5A, the processing movement B is imparted on the packages 2 by two curved rails 55, between which the packages 2 and the upper parts 51 of the cells 13 are moved from the starting position A to the end position E. The upper part 51 of the cell 13 and the package 2 in FIG. 5A are shown in a position displaced to the left as a result of the positive contact with the curved rails 55. The same applies to the embodiment according to FIG. 5B in which the upper part 52 and the lower part 49 of the cell 13 are connected to each other by a spring means 56. In addition, only one curved rail 57 is shown, which deflects the upper part 52 of the cell 13 to the left, counter to the restoring force of the spring means 56. The restoring force of the spring means 56 ensures that the upper part 52 of the cell 13 remains in contact with the curved rail 57 and returns to a starting position. The second curved rail is thus unnecessary. FIG. 5C illustrates the case in which an electrical device 58 generates a magnetic field M by applying a corresponding voltage through which the upper part 53 of the cell 13 is moved and thereby deflected in the processing movement B. The lower part 50 of the cell 13 is moved independently therefrom in the transporting direction T in a straight line from the starting position A to the end position E. In the schematic plan view according to FIG. 5D, in which the principle described for FIGS. SA-C for the impingement of the packages 2 with a processing movement B can be seen, the displacement of the packages 2 in a processing movement B transverse to the transport direction T against a restoring force of a spring means 56 triggered by a curved rail 57 according to FIG. 5B is shown. The packages 2 are thereby moved from the starting position A to the end position E.

If necessary, the cells 13, together with the packages 2, can also be stopped between the starting position A and the end position E, in order then to be impinged with a processing movement B in a processing position. This can be done as in FIG. 6A-B by a cam 59 which is mounted on a cam disc 60. The cam disc 60 shown in FIGS. 6A-B at two different times rotates and thereby comes into positive contact with an upper part 61 of the cell 13, which is thereby displaced outwardly relative to the lower part 62 of the cell 13, in order to effect a processing movement B of the package 2 incorporated in the upper part 61 of the cell 13. In this case, the upper part 61 and the lower part 62 of the cell 13 may be connected via a spring means whose restoring force holds the upper part 61 of the cell 13 in contact with the cam 59 and moves the package 2 back to a starting position after the deflection. Subsequently, the package 2 is moved further in the transport direction T into the end position E. FIG. 7 shows that the same cam disc 60 as in FIGS. 6A-B can cause tilting of the upper part 63 of the cell 13, which has the package 2, in order to transfer a processing movement B, designed as a tilting movement, to the package 2. The upper part 63 of the cell 13 is then not displaced by the cam 59 of the cam disc 60 with respect to the lower part 64 of the cell 13 but is instead tilted.

### LIST OF REFERENCE NUMERALS

1 Filling machine
2 Package
3 Device
4 Bundle
5 Package blank
6 Packaging sleeves
7 Feed device
8 Mandrel
9 Mandrel wheel
10 Heating unit
11 Pre-folding
12 Press
13 Cells
14 Transport device
15 Aseptic chamber
16 Sterilisation zone
17 Filling and sealing zone
18 Sterile air connection
19 Preheating device
20 Sterilising device
21 Drying device
22 Filling device
23 Closing device
24 Nozzle
25 Compressor
26 Filter
27 Heating device
28 Pump
29 Vaporiser
30 Nozzle
31 Compressor
32 Filter
33 Compressor
34 Filter
35 Heating device
36 Cell
37 Reservoir
38 Nozzle
39 Lower part
40 Upper part
41 Curved rail
42 Curved rail
43 Upper part
44 Lower part
45 Lower part
46 Upper part
47 Curved rail
48-50 Lower part
51-53 Upper part
54 Rail system
55 Curved rail
56 Spring means
57 Curved rail
58 Electrical device
59 Cam
60 Cam disc
61 Upper part
62 Lower part
63 Upper part
64 Lower part
A Starting position
B Processing movement
E End position
T Transport direction

1.-15. (canceled)

16. A method for filling packages open on one side with flowable products in a filling machine comprising the steps of:
introducing hot air, drying air, a sterilising agent and/or a flowable product is via at least one nozzle into packages open on one side;
moving the packages from a starting position, before the introduction of the hot air, the drying air, the sterilising agent and/or the flowable product, into an end position, after the introduction of the hot air, the drying air, the sterilising agent and/or the flowable product; and
moving the packages, during the introduction of the hot air, the drying air, the sterilising agent and/or the flowable product, along a straight or continuously curved transport direction defined by the starting position and the end position and are moved in a not exclusively vertical processing movement overlaying the transport direction and extending at least in sections transversely to the transport direction.

17. The method according to claim 16, further comprising the step of:
moving the packages respectively on a carrier and/or respectively in a cell of a transport device from the starting position to the end position, in particular through the filling machine, and in which, preferably, the carriers and/or the cells for effecting the processing movement of the packages are moved at least in sections in at least one direction transverse to the transport direction, in particular back and forth.

18. The method according to claim 16, further comprising the steps of:
tilting the packages; and
tilting the packages in a plane parallel to the transport direction and/or in a plane perpendicular to the transport direction.

19. The method according to claim 16, further comprising the steps of:
moving packages transverse to the transport direction; and
moving the packages in a horizontal plane and/or in a vertical plane.

20. The method according to claim 17, further comprising the step of:
moving a part of the carriers and/or cells from the starting position into the end position at least substantially exclusively in the transport direction, wherein a part of the carriers and/or cells associated with the packages or connected to the packages carries out the processing movement.

21. The method according to claim 17, wherein
at least one part of the carriers and/or cells comes into positive contact against a curved rail and/or a cam between the starting position and the end position and consequently carries out the processing movement.

22. The method according to claim 17, further comprising the step of:
exposing at least one part of the carriers and/or cells to a magnetic field between the starting position and the end position that consequently carries out the processing movement.

23. The method according to claim 17 further comprising the step of:
connecting parts of the carriers and/or cells to one another via a spring means and that parts of the carriers and/or cells that are retracted by the spring means back into a starting position after a deflection.

24. A filling machine for filling packages open on one side with flowable products according to claim 16, wherein at least one nozzle is provided for introducing hot air, drying air, sterilising agent and/or flowable product into the packages open on one side and wherein a transport device is provided for transporting the packages open on one side from a starting position, before the introduction of the hot air, the drying air, the sterilising agent and/or the flowable product, into an end position, after the introduction of the hot air, the drying air, the sterilising agent and/or the flowable product, along a straight or continuously curved transport direction defined by the starting position and the end position, wherein means are provided for moving the packages during the introduction of the hot air, the drying air, the sterilising agent and/or the flowable product in a not exclusively vertical processing movement overlaying the transport direction and extending at least in sections transversely to the transport direction.

25. The filling machine according to claim 24, wherein
the transport device has carriers and/or cells for holding and moving the packages from the starting position to the end position, and the carriers and/or cells are configured to effect the processing movement of the packages at least in sections in at least one direction transverse to the transport direction, in particular back and forth.

26. The filling machine according to claim 24, wherein
means are provided for tilting the packages and in that the means are provided for tilting the packages in a plane parallel to the transport direction and/or in a plane perpendicular to the transport direction.

27. The filling machine according to claim 24, wherein
means are provided for moving the packages transverse to the transport direction, and in that the means are provided for moving the packages in a horizontal plane and/or in a vertical plane.

28. The filling machine according to claim 24, wherein
the carriers and/or cells are designed in at least two parts and the at least two parts are connected displaceably and/or tiltably relative to one another with at least one rail system at least one flexible joint and/or at least one spring means.

29. The filling machine according to claim 24, wherein
at least one curved rail and/or at least one cam is provided for positive contact with at least one part of the carriers and/or cells between the starting position and the end position, such that the processing movement is affected.

30. The filling machine according to claim 24, wherein
at least one device is provided for generating a magnetic field passed by the carriers and/or cells for effecting the processing movement.

31. The filling machine of claim 24, wherein the filling packages open on one side are cardboard composite packages, and wherein the flowable products are food.

32. The filling machine according to claim 30, wherein the at least one device is an electrical device.

33. The filling machine according to claim 26, wherein means are provided for tilting packages back and forth together with at least parts of the carriers and/or cells and, in that, the means are provided for tilting the packages together with at least parts of the carriers and/or cells in a plane parallel to the transport direction and/or in a plane perpendicular to the transport direction.

34. The filling machine according to claim 27, wherein means are provided for moving the packages transverse to the transport direction together with at least parts of the carriers and/or cells back and forth, and in that, the means are provided for moving the packages together with at least parts of the carriers and/or cells in a horizontal plan and/or in a vertical plane.

35. The method according to claim 18, further comprising the steps of:
tilting the packages together with at least parts of the carriers and/or cells back and forth; and
tilting the packages together with at least parts of the carriers and/or cells back and forth between a plane parallel to the transport direction and/or in a plane perpendicular to the transport direction.
